SEQUENCE LISTING (25 sequences)

SEQ ID NO: 1 - DNA, 1915 bp, Homo sapiens
  misc_feature: SCC-S2
ctcccgccggctctaacccgcgcttggctaaggtccgcgggaacccgtgagccaccgagagagcagagaactcggcgccgccaaacagcccagctcgcgcttcagcgtcccggcgccgtcgccgactcctccgatggccacagatgtctttaattccaaaaacctggccgttcaggcacaaaagaagatcttgggtaaaatggtgtccaaatccatcgccaccaccttaatagacgacacaagtagtgaggtgctggatgagctctacagagtgaccagggagtacacccaaaacaagaaggaggcagagaagatcatcaagaacctcatcaagacagtcatcaagctggccattctttataggaataatcagtttaatcaagatgagctagcattgatggagaaatttaagaagaaagttcatcagcttgctatgaccgtggtcagtttccatcaggtggattatacctttgaccggaatgtgttatccaggctgttaaatgaatgcagagagatgctgcaccaaatcattcagcgccacctcactgccaagtcacatggacgggttaataatgtctttgatcatttttcagattgtgaatttttggctgccttgtataatccttttgggaattttaaaccccacttacaaaaactatgtgatggtatcaacaaaatgttggatgaagagaacatatgagcacatgagttaagattgtgactgatcatgatttatttgaagatggagcactgctgatttatgaaggaaaaaagaagaattttctaaagattacacatatttcagaaagactttacccaattcagttgtcagacataatgatttatttgaaggcttgttttatttgaagaaaagcatattgccaaaaattctggttaaaagcttcctaatgggtaacagaccatgggagagatatgtggttgggtaatgcgaatgtagttatacaaagaaaaatacagatgtctccagacctgaggactttttaatagggcggttgttgtgttggtggcacattggatatttctaacatgtacaaagctatgtattttgatttactttcatttcttgctatgtatatgtacttttcttaaaatgccaagaactttctcttgctatcattgctccttttgaaacaattcaattttcatgtctacagctgactgttttgttaagattgagtcatcgacattcaggatttaagtctgaggtagtcaaccctcaggaaaaaaaaaatggcttatctgaaatcagtactgtggaaatgaactatattagctattatgaataatgtccagtataagaatatgcttctggaattgagttctccttttaagtaccaatgatacttaaatttctcagaaatgtaatggtgtgtcattgccttgaaatgcttgcttagggcttcttttatgttatcttaaaaagtgctggtgaattttccattttttacatccatttcacatgtaagagacaaaaaagtctagattggtcttgatattgagataataaaaagtaagtagcattaagaaaggtaacaatcttcattctacagatgaactcattgaaacaatttaggggaatgaggggcaaaaggggagaaatactgctaaagaacatgagcataaaaatgcgtgcgtttcagtgtttaagaaggcttgataaagaatgtcacttttttatttaactgataagatttttgttattttttactttgataagtaaaccaaagaatatttgtatttcaagcagtttgtgtggtgtttctatataattttctgtgtataaataataaagtaggcatttgtttattttgtaaaaaagaaatgaaaatctgctggccagctatgtcctctaggaaatgacagacccaaccaccagcaataaacatttccattg

SEQ ID NO: 2 - protein, 188 aa, Homo sapiens
  MISC_FEATURE: SCC-S2
MATDVFNSKNLAVQAQKKILGKMVSKSIATTLIDDTSSEVLDELYRVTREYTQNKKEAEKKIKNLIKTVIKLAILYRNNQFNQDELALMEKFKKKVHQLAMTVVSFHQVDYTFDRNVLSRLLNECREMLHQIIQRHLTAKSHGRVNNVFDHFSDCEFLAALYNPFGNFKPHLQKLCDGINKMLDEENI

SEQ ID NO: 3 - protein, 66 aa, Homo sapiens
  MISC_FEATURE: SCC-S2 - fragment
DDTSSEVLDELYRVTREYTQNKKEAEKIIKNLIKTVIKLAILYRNNQFNQDELALMEKFKKKVHQL

SEQ ID NO: 4 - protein, 59 aa, Mus musculus
  MISC_FEATURE: CASH Alpha/Beta - fragment
NDVSSLVFLTRITRDYTGRGKIAKDKSFLDLVIELEKLNLIASDQLNLLEKCLKNIHRI

SEQ ID NO: 5 - protein, 56 aa, Homo sapiens
  MISC_FEATURE: CASH Alpha/Beta - fragment
SDVSSLIFLMKDYMGRGKISKEKSFLDLVVELEKLNLVAPDQLDLLEKCLKNIHRI

SEQ ID NO: 6 - protein, 56 aa, Mus musculus
  MISC_FEATURE: FLIP (L) - fragment
NDVSSLVFLTRDYTGRGKIAKDKSFLDLVIELEKLNLIASDQLNLLEKCLKNIHRI

SEQ ID NO: 7 - protein, 56 aa, Homo sapiens; identical to SEQ ID NO: 5
  MISC_FEATURE: FLIP (L) - fragment

SEQ ID NO: 8 - protein, 56 aa, Mus musculus
  MISC_FEATURE: FLICE (Casp8) - fragment
LELRSFKFLLNNEIPKCKLEDDLSLLEIFVEMEKRTMLAENNLETLKSICDQVNKS

SEQ ID NO: 9 - protein, 56 aa, Homo sapiens
  MISC_FEATURE: FLICE (Casp8) - fragment
SELRSFKFLLQEEISKCKLDDDMNLLDIFIEMEKRVILGEGKLDILKRVCAQINKS

SEQ ID NO: 10 - protein, 53 aa, Homo sapiens
  MISC_FEATURE: SCC-S2 - fragment
SSEVLDELYRVTREYTQNKKEAEKIIKNLIKTVIKLAILYRNNQFNQDELALM

SEQ ID NO: 11 - protein, 51 aa, Homo sapiens
  MISC_FEATURE: Poliovirus 1 VP1 - fragment
TQQISDKITELTNMVTSTITEKLLKNLIKIISSLVIITRNYEDTTTVLATL

SEQ ID NO: 12 - protein, 51 aa, Homo sapiens
  MISC_FEATURE: Poliovirus 2 Polyprotein - fragment
TQQIGDKVSELTSMVTSTITEKLLKNLIKIISSLVIITRNYEDTTTVLATL

SEQ ID NO: 13 - protein, 51 aa, Homo sapiens
  MISC_FEATURE: Poliovirus 3 Polyprotein - fragment
TQQIGDKISELTSMVTSTITEKLLKNLIKIISSLVIITRNYEDTTTVLATL

SEQ ID NO: 14 - protein, 51 aa, Homo sapiens; identical to SEQ ID NO: 11
  MISC_FEATURE: Poliovirus 1 P2-3b - fragment

SEQ ID NO: 15 - protein, 52 aa, Homo sapiens
  MISC_FEATURE: SCC-S2 - fragment
SSEVLDELYRVTREYTQNKKEAEKIIKNLIKTVIKLAILYRNNQFNQDELAL

SEQ ID NO: 16 - protein, 53 aa, Vaccinia virus
  MISC_FEATURE: DNA Polymerase - fragment
SSNSKSVPERINKGTSETRRDVSKFHKNMIKTYKTRLSEMLSEGRMNSNQVCI

SEQ ID NO: 17 - protein, 42 aa, Homo sapiens
  MISC_FEATURE: SCC-S2 - fragment
TLIDDTSSEVLDELYRVTREYTQNKKEAEKIIKNLIKTVIKL

SEQ ID NO: 18 - protein, 46 aa, Canine adenovirus
  MISC_FEATURE: DNA Pol - fragment
TLIPDTRTTVFPEWKCLAREYVQLNISAKEEADKSKNQTMRSIAKL

SEQ ID NO: 19 - protein, 54 aa, Homo sapiens
  MISC_FEATURE: SCC-S2 - fragment
KKEAEKIIKNLIKTVIKLAILYRNQFNQDELALMEKFKKKVHQLAMTVVSFHQV

SEQ ID NO: 20 - protein, 55 aa, Homo sapiens
  MISC_FEATURE: Alpha1 (E) - Catenin - fragment
AKKIAEAGSRMDKLGRTIADHCPDSACKQDLLAYLQRIALYCHQLNICSKVKAEV

SEQ ID NO: 21 - protein, 55 aa, Homo sapiens
  MISC_FEATURE: Alpha2 (E) - Catenin - fragment
AKKIAEAGSRMDKLARAVADQCPDSACKQDLLAYLQRIALYCHQLNICSKVKAEV

SEQ ID NO: 22 - protein, 55 aa, Homo sapiens
  MISC_FEATURE: Vinculin - fragment
AKDIAKASDEVTRLAKEVAKQCTDKRIRTNLLQVCERIPTISTQLKILSTVKAIM

SEQ ID NO: 23 - DNA, 27 bp, Artificial
  Primer
cccaagcttctcccgccggctctaacc

SEQ ID NO: 24 - DNA, 57 bp, Artificial
  Primer
ccaggaattctcacttgtcatcgtcgtccttgtagtctatgttctcttccatccaac

SEQ ID NO: 25 - protein, 17 aa, Artificial
  SCC-S2 Fragment
CYRNNQFNQDELALMEK

(19) United States
(12) Patent Application Publication
(10) Pub. No.: US 2010/0041142 A1
(43) Pub. Date: Feb. 18, 2010

(54) ANTI-APOPTOTIC GENE SCC-S2 AND DIAGNOSTIC AND THERAPEUTIC USES THEREOF

(75) Inventors: Usha Kasid, Rockville, MD (US); Deepak Kumar, Annandale, VA (US); Prafulla Gokhale, Superior, CO (US); Imran Ahmad, Wadsworth, IL (US)

Correspondence Address: PILLSBURY WINTHROP SHAW PITTMAN, LLP, P.O. BOX 10500, MCLEAN, VA, 22102, US

(73) Assignee: Georgetown University, Washington, DC (US)

(21) Appl. No.: 12/467,802

(22) Filed: May 18, 2009

Related U.S. Application Data
(63) Continuation of application No. 11/600,437, filed on Nov. 16, 2006, now abandoned.
Application No. 11/600,437 is a division of application No. 10/627,571, filed on Jul. 25, 2003, now abandoned.
Application No. 10/627,571 is a continuation of application No. PCT/US02/02212, filed on Jan. 28, 2002.
(60) Provisional application No. 60/264,062, filed on Jan. 26, 2001.

(51) Int. Cl. C12N 5/02

(52) U.S. Cl. 435/375

(57) ABSTRACT

A gene that is a positive mediator of tumor growth and metastasis in certain cancer types is provided. This gene and corresponding polypeptide have diagnostic and therapeutic application for detecting and treating cancers that involve expression of SCC-S2 such as renal, ovarian, head and neck, breast, prostate, brain, chronic myelogenous leukemia, lung, lymphoblastic leukemia, and colorectal adenocarcinoma cells.

### RELATED APPLICATIONS

The present application is a continuation of co-pending international patent application PCT/US02/02212, filed Jan. 28, 2002, and which designates the United States, and which claims priority to U.S. Provisional Application Ser. No. 60/264,062, filed Jan. 26, 2001, the contents of both of which are incorporated herein by reference in their entirety.

### FIELD OF THE INVENTION

The present invention relates to a gene that encodes a polypeptide that negatively mediates apoptosis. This polypeptide is a useful target for identifying compounds that modulate cancer progression by inhibiting apoptosis. Also, this polypeptide is useful as a diagnostic target for detecting cancers wherein this polypeptide is overexpressed, e.g., renal and ovarian cancers and leukemias.

### BACKGROUND OF THE INVENTION

Increasing evidence suggests that apoptosis requires activation of members of the ICE¹-like family of cysteine proteases, also known as caspases. The caspase activation appears to be triggered by some members of the TNFR ¹superfamily, including TNF receptors, TNFR1 (p55/CD120a) and TNFR2 (p75/CD120b), and Fas/Apo-1 (CD95). TNF binds to TNFR1, and FasL binds to Fas. TNFR1 and Fas, also known as death receptors, are characterized by the presence of a cytoplasmic sequence motif called the death domain (DD), which interacts with the DD of the adaptor molecules FADD and TRADD, recruiting them to the membrane. TRADD interacts with FADD, and FADD, in turn, associates with an apical caspase, FLICE (caspase 8/MACH/Mch5) through death-effector-domains (DEDs) present at the carboxy-terminus of FADD and the amino-terminus of FLICE, resulting in the assembly of a receptor-associated death-inducing signaling complex (DISC). DISC-associated FLICE signals proteolytic activation of downstream caspases, ultimately leading to apoptosis (reviewed in ref. #1). FADD mutant containing only the DD or FLICE containing two DEDs can act as a dominant negative inhibitor of apoptosis (24). Because ligand activation of a death receptor does not lead to apoptosis in all cell types, it has been suggested that natural cell death inhibitory molecules may exist in certain cells. Indeed, FLICE-inhibitory proteins (FLIP/CASH/1-FLICE/FLAME-1) containing two sequence motives with significant homology to DEDs have been identified (5-9). FLIPs contain two DEDs in the amino-terminus, and are represented by two splice variants: FLIP(L), the long form, and FLIP(S), the short form. Carboxy-terminal extension of the longer variant shows homology to the caspase-like protease homology domain, but lacks active-site cysteine, suggesting that it is devoid of proteolytic activity. These proteins bind to FLICE through DEDs, blocking the binding and proteolytic activation of effector caspases. Consistent with these findings, a viral homologue of cellular FLIP (v-FLIP) identified in herpes and molluscum contagiosum viruses exhibits anti-apoptotic activity, and overexpression of cellular FLIP suppresses FasL and TNF-α-induced apoptosis (5, 10-12). ¹The abbreviations used in this Application are: ICE, interleukin-1β-converting enzyme; TNFR, tumor necrosis factor receptor; TNF-α, tumor necrosis factor-α; FADD, Fas-associated death domain; TRADD, TNF receptor-associated death domain; FLICE, FADD-like ICE; DD, Death Domain; DED, Death-effector-domain; FLIP, FLICE-inhibitory protein; HNSCC, head and neck squamous cell carcinoma; PCR, polymerase chain reaction.

Several reports indicate that negative regulators of apoptosis, including the FLIP family of proteins may also trigger tumorigenesis in appropriate cells (8, 13). For example, increased expression of FLIP has been found in Fas ligand-resistant melanoma cell lines and in metastatic cutaneous melanoma lesions from patients, whereas no expression was detected in melanocytes surrounding the hair follicle of the skin (8). Second, activation of the Ras/Raf-1/MKK/MAPK pathway is known to play major roles in tumorigenesis and protection against cytotoxic agents (reviewed in ref. #s 14 and 15), and activation of MKK1 has been shown to abrogate Fas-initiated apoptosis through the induction of FLIP expression (16).

### OBJECTS AND SUMMARY OF THE INVENTION

It is an object of the invention to provide a novel gene that encodes a polypeptide which is a negative mediator of apoptosis.

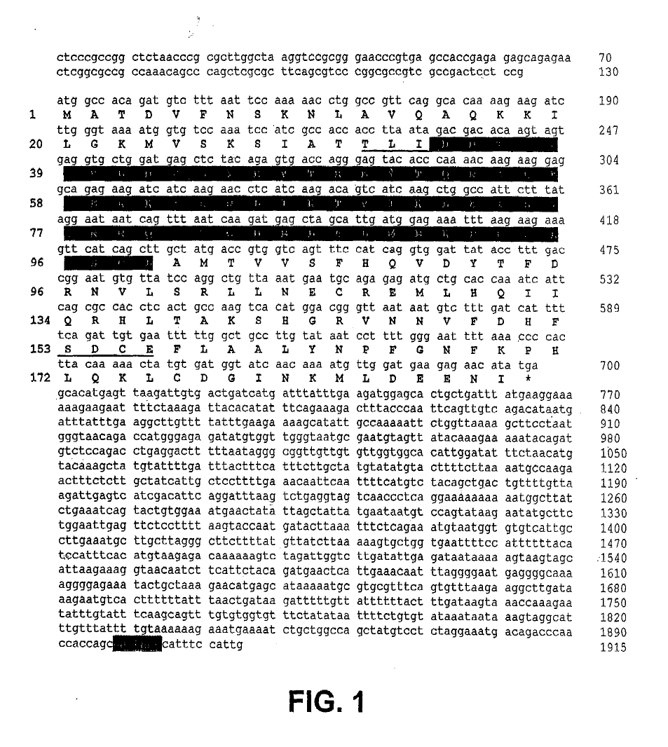

It is a more specific object of the invention to provide an SCC-S2 nucleic acid sequence encoding the polypeptide identified in FIG. 1 having SEQ ID NO: 2, or a homolog or analog thereof, that encodes a polypeptide having at least 90% sequence identity to said polypeptide, or a fragment thereof that encodes a polypeptide that negatively mediates apoptosis.

It is another specific object of the invention to provide a nucleic acid sequence corresponding to nucleotides 397 to 1915 of SEQ ID NO: 1 contained in FIG. 1 or a fragment thereof which is at least 100 nucleotides in length.

It is another object of the invention to provide a SCC-S2 polypeptide that negatively mediates apoptosis having the amino acid sequence contained in SEQ ID NO: 2, which sequence is depicted in FIG. 1, or a fragment thereof which is at least 50 amino acids in length or an analog or homolog having at least 90% sequence identity to said polypeptide which negatively mediates apoptosis.

It is another object of the invention to provide an antibody that specifically binds SCC-S2 polypeptide.

It is another specific object of the invention to provide a method for identifying compounds that promote apoptosis by screening for compounds that specifically bind SCC-S2 polypeptide.

It is another specific object of the invention to provide a method for detecting or evaluating the prognosis of a cancer characterized by overexpression of SCC-S2 by detecting expression of SCC-S2 in an analyte obtained from a patient tested for cancer and correlating the level of expression to a positive or negative diagnosis for cancer.

It is another object to provide a method of treating or preventing a cancer characterized by overexpression of SCC-S2 comprising administering a compound that inhibits SCC-S2 gene expression and/or activity of SCC-S2 polypeptide.

It is yet another object to provide a method for treating cancer comprising administering at least one antisense oligonucleotide or ribozyme that inhibits SCC-S2 expression, thereby inhibiting cancer cell proliferation and/or metastatic potential.

It is still another object of the invention to provide a pharmaceutical composition for treatment of cancer that comprises an antagonist of SCC-S2 expression and/or activity and a pharmaceutically acceptable carrier. Preferably, such compositions will comprise liposomal formulations.

Another object of the invention is to provide diagnostic compositions for detection of cancer that comprise an oligonucleotide that specifically binds SCC-S2 DNA or an antibody that specifically binds the SCC-S2 polypeptide, attached directly or indirectly to a label, and a diagnostically acceptable carrier.

It is another object of the invention to provide methods for inhibiting tumor growth and/or metastasis by administration of a molecule that antagonizes the expression and/or activity of SCC-S2.

It is a preferred object of the invention to provide liposomal formulations for antisense therapy that inhibit tumor growth and/or metastasis which comprise antisense oligonucleotides specific to SCC-S2, optionally in association with cytotoxic moieties such as radionuclides, radiation, anticancer drugs, other biological agents including DNA, RNA, proteins and antibodies.

### DETAILED DESCRIPTION OF THE FIGURES

FIG. 1: This figure contains a cDNA and predicted amino acid sequence for SCC-S2. Nucleotide sequences of a cDNA clone (1519 bp, nucleotides 397-1915) isolated from a human heart cDNA library using a 259 bp cDNA probe (large box), and an overlapping EST clone (nucleotides 1-396) are shown. Nucleotide positions are indicated by numbers on the right. Predicted longest ORF (188 amino acids) is shown. Amino acid positions are numbered on the left. The polyA+ signal sequence is shown in bold in a small box. The proposed main structural features of the SCC-S2 protein are: putative DED, shaded; and Protein Kinase C and Casein Kinase II phosphorylation-sites, bolded and underlined, respectively. The nucleotide sequence is reported in the GenBank DNA database (accession numbers: AA406630 (nucleotides 1-396), AF098933 (nucleotides 397-911), U68132 (nucleotides 912-1170), and AF098934 (1171-1915)).

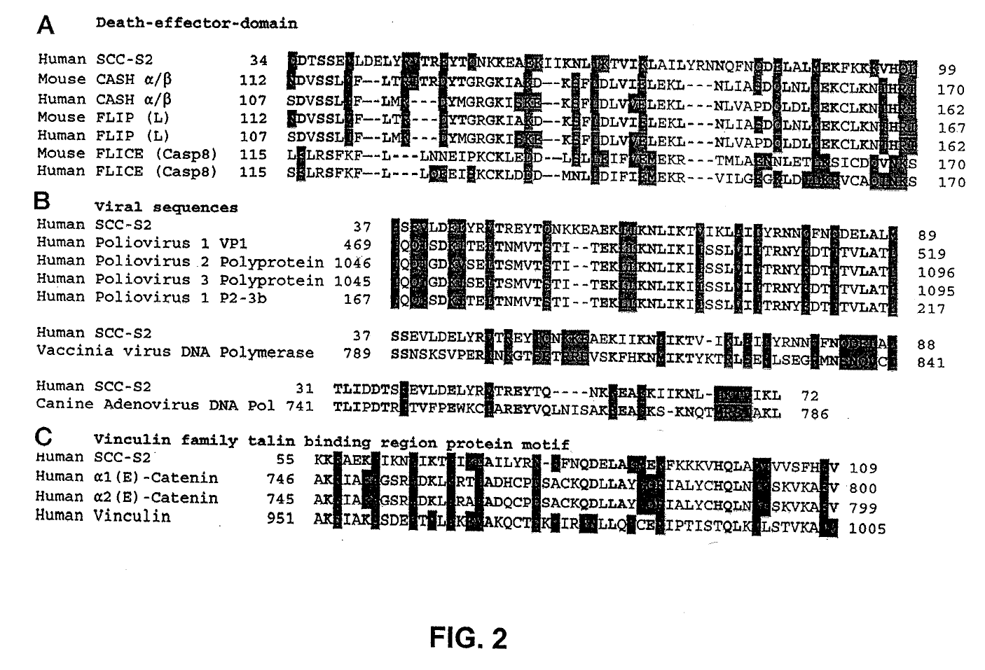

FIG. 2: This figure contains alignments of the amino acid sequences for the putative functional domains of SCC-S2. Positions of the amino acids at the left and right ends of each sequence are shown. Dashes indicate gaps inserted in the sequence to allow optimal alignment. Amino acids that are identical to SCC-S2 are shown in bold type, and amino acids that are similar are shaded.

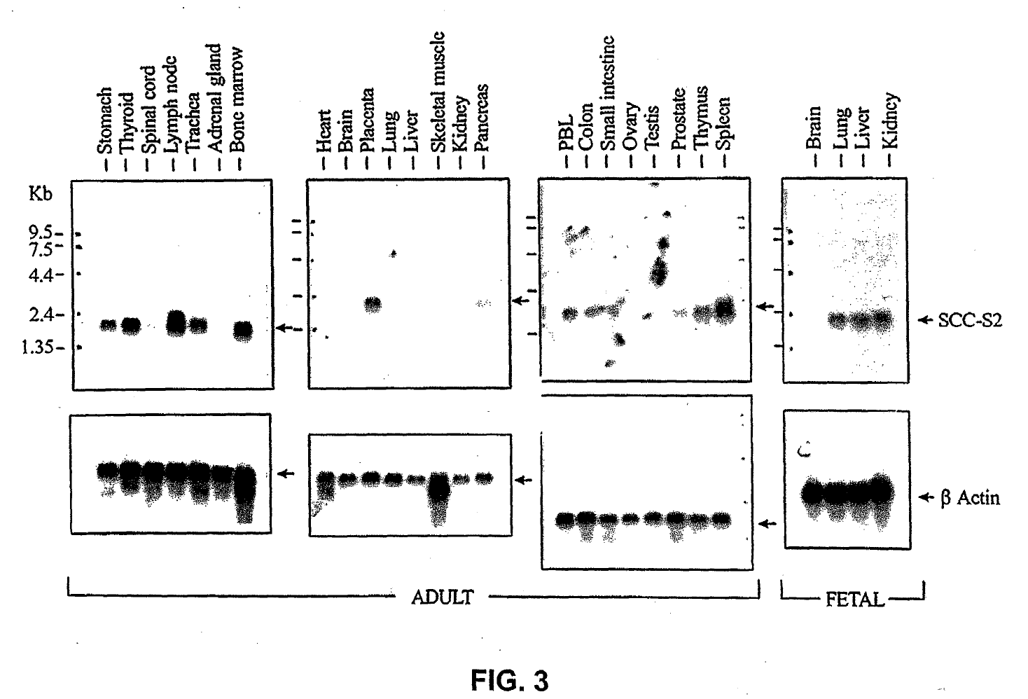

FIG. 3: This figure shows normal tissue distribution of SCC-S2 gene expression. Human adult and fetal tissue RNA blots (Clontech) were probed with a radiolabeled ˜1.5 kb SCC-S2 cDNA fragment. The blots were reprobed with β actin cDNA. Auoradiographs were scanned using a software program (Image Quant, Molecular Dynamics, Inc.), and SCC-S2 expression was normalized to p actin in the corresponding lane.

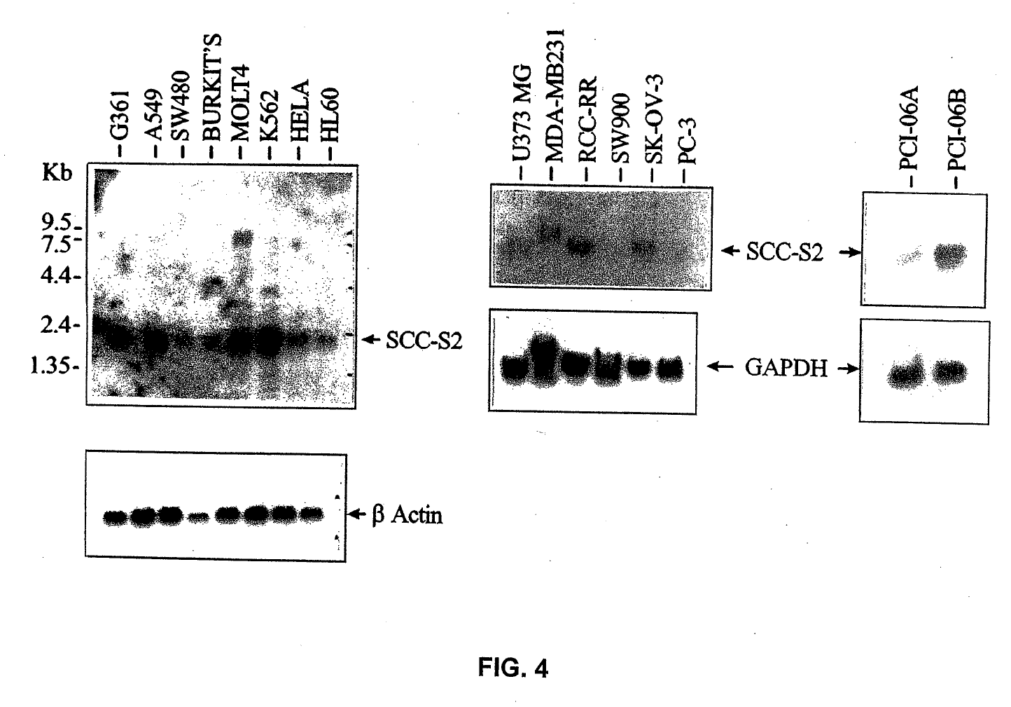

FIG. 4: This figure shows expression of SCC-S2 transcript in human cancer cell lines. Left panel, cancer cell line blot (Clontech) was probed with a radiolabeled ˜1.5 kb SCC-S2 cDNA fragment and reprobed with β actin cDNA. Middle and right panels, blots were sequentially hybridized to ˜1.5 kb SCC-S2 cDNA and GAPDH cDNA probes. Auoradiographs were computer-scanned, and SCC-S2 mRNA expression was normalized to β actin or GAPDH. G361, melanoma; A549, lung carcinoma; SW480, colorectal adenocarcinoma; MOLT4, lymphoblastic leukemia; K562, chronic myelogenous leukemia; HL60, promyelocytic leukemia; U373MG, glioblastoma; MDA-MB231, breast carcinoma; RCC-RR, renal cell carcinoma; SW900, lung carcinoma; SKOV-3, ovarian carcinoma; PC-3, prostate carcinoma, and PCI-06A and PCI-06B, head and neck squamous cell carcinoma.

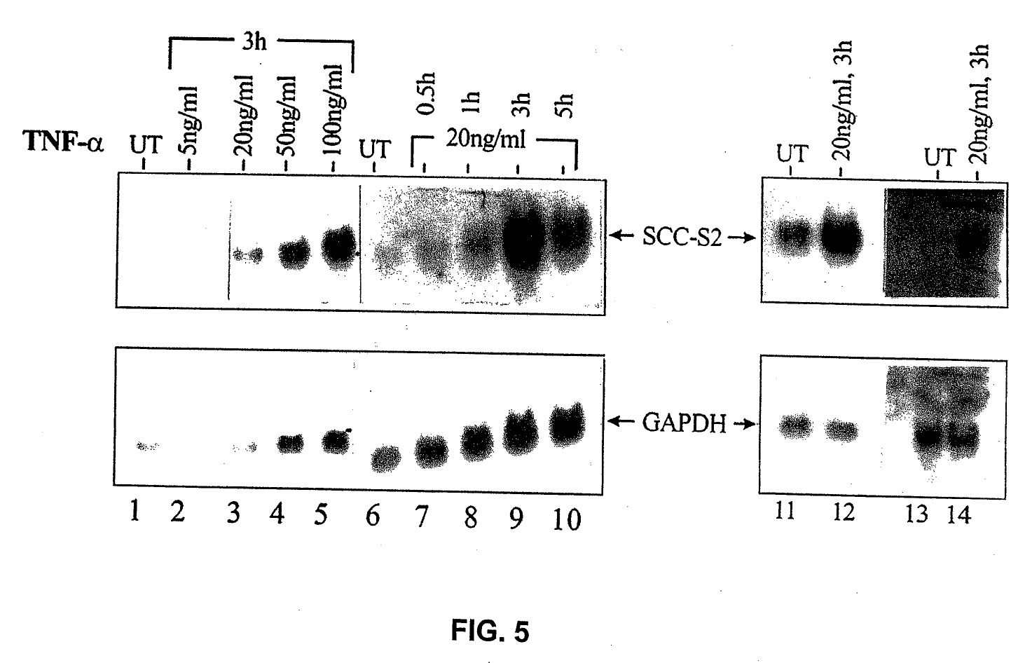

FIG. 5. This figure shows that TNF-α stimulates the steady state level of SCC-S2 mRNA. Logarithmically growing cells including the control cells were switched to serum-free medium 2 h prior to the addition of indicated concentration of TNF-α, followed by incubation for various times. Total RNA blots were sequentially hybridized to radiolabeled ˜1.5 kb SCC-S2 cDNA and GAPDH cDNA probes. Autoradiographs were computer-scanned and SCC-S2 expression was normalized to the corresponding GAPDH signal. Lanes 1-10, A549 lung carcinoma cells; 11 and 12, SKOV-3 ovarian carcinoma cells; and 13 and 14, PCI-04A laryngeal squamous carcinoma cells.

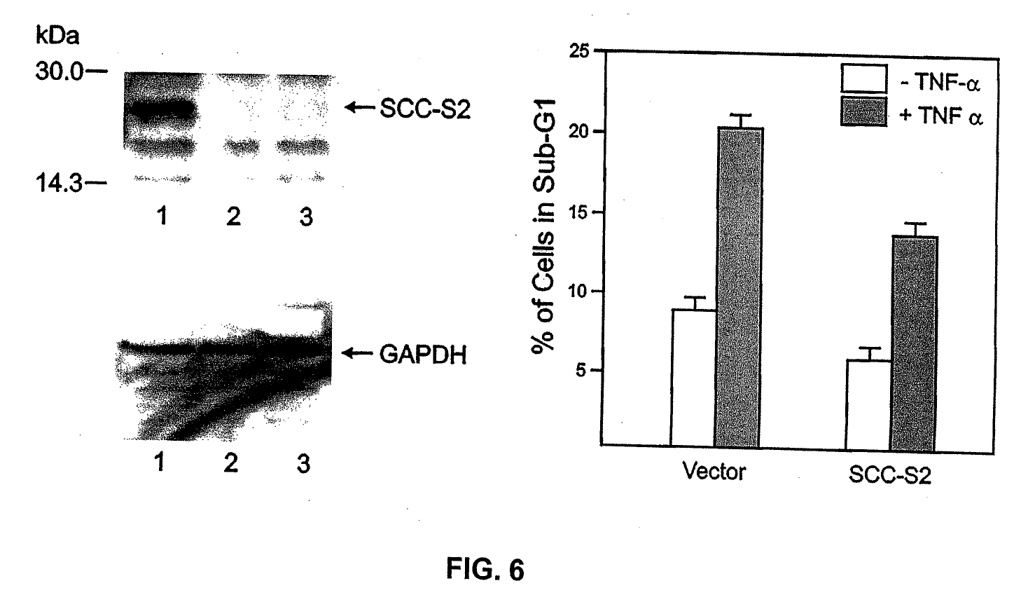

FIG. 6. This figure shows that expression of exogenous SCC-S2 protein is associated with decreased apoptosis. Left panel, HeLa cells were transiently transfected with FLAG epitope-tagged SCC-S2 cDNA (lane 1), or vector (lane 2), followed by immunoblotting with FLAG-M2 antibody (Top). The same blot was reprobed with anti-GAPDH antibody (Bottom). Lane 3, untransfected. Right panel, 30 h after transfection of HeLa cells, medium was switched to the medium containing 1% FBS (1 h). TNF-α (100 ng/ml) was added and incubations continued for 4 h, followed by the FACS analysis as described in the examples. A representative experiment performed in triplicate is shown.

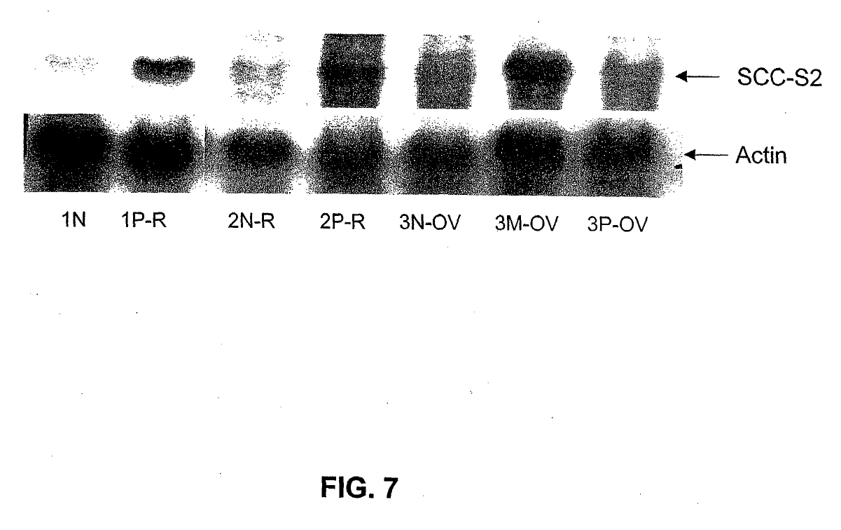

FIG. 7. This figure shows steady state expression levels of SCC-S2 mRNA in normal adjacent (N), primary tumor (P) and metastatic tumor (M) tissues. Tissue specimens from three patients (1, 2, 3) were examined. Northern blots were sequentially probed with radiolabeled SCC-S2 cDNA, followed by β-actin cDNA.

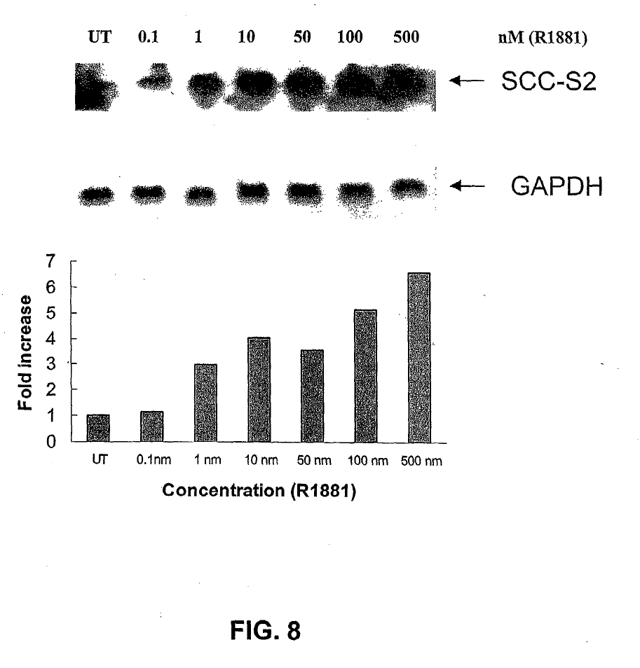

FIG. 8. This figure shows that androgen induces the SCC-S2 mRNA level in LnCaP prostate cancer cells. The relative (fold) increase in mRNA level was calculated after normalizing the data with GAPDH signal in the corresponding lane as an internal control.

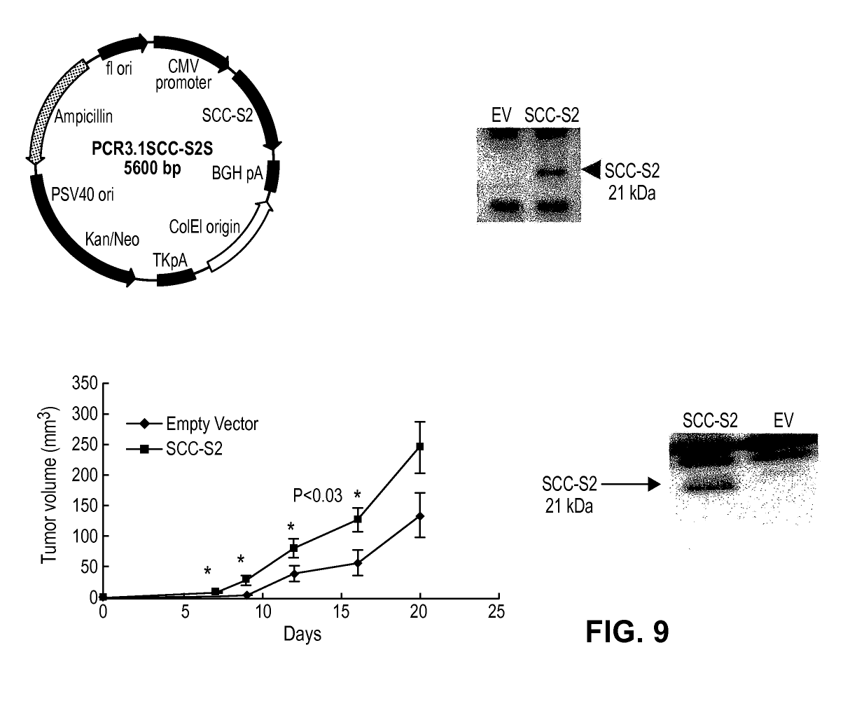

FIG. 9. This figure shows the effect of expression of SCC-S2 on MDA-MB 435 tumor growth. MDA-MB 435 cells were transfected with FLAG-tagged SCC-S2 cDNA (SCC-S2) or expression vector (EV) (top, left). Anti-FLAG antibody was used to detect the expression of exogenous SCC-S2 protein in transfected cells (top, right). Female athymic mice were inoculated with MDA-MB 435 transfectomas and that growth was monitored. Data shown are mean time volume±SE (n=s: bottom, left). Expression of SCC-S2 in tumor xenografts was confirmed by immunoblotting with anti-FLAG antibody (arrow; bottom, right).

### BRIEF DESCRIPTION OF THE INVENTION

The molecular genetic factors that negate cell death and contribute to tumor growth and metastasis can be attractive targets for therapeutic intervention. In a search for such genes, the present inventors earlier identified a very small portion of a cDNA based on a relatively higher steady state level of its mRNA in a metastatic head and neck squamous cell carcinoma (HNSCC)-derived cell line (PCI-06B) as compared to its matched (from the same patient) primary tumor-derived cell line (PCI-06A) [see Patel et al., “Identification of seven differentially displayed transcripts in human primary and matched metastatic head and neck squamous cell carcinoma cell lines: Implications in metastasis and/or radiation response”, Oral Oncol. Eur. J. Cancer 33:193-199, (1997)].

By contrast, the present invention provides a full length cDNA encoding a gene which was named as SCC-S2 that is a negative mediator of apoptosis [see Kumar et al., “Identification of a novel tumor necrosis factor-a-inducible gene, SCC-S2, containing the consensus sequence of a death effector domain of Fas-associated death domain-like interleukin-1β-converting enzyme-inhibitory protein”, J. Biol. Chem. 275: 2973-2978 (2000), which reference is incorporated by reference herein]. In this paper and as described herein, the inventors showed that SCC-S2 mRNA expression is transiently induced following exposure of cells to TNF-α, a cytokine known to trigger diverse cellular responses through TNF receptors, TNFR1 and TNFR2. Transient transfection experiments using FLAG-tagged expression vector containing SCC-S2 cDNA indicate that SCC-S2 is a negative mediator of apoptosis. Enhanced cell proliferation and tumorigenecity of hormone-independent breast cancer cells stably transfected with SCC-S2 cDNA (data shown below) has been observed. SCC-S2-specific peptides have been designed and antibodies generated (please see below). Liposome-entrapped SCC-S2 antisense oligonucleotide (LES2AON) or phosphorothioated SCC-S2 antisense oligonucleotides are being developed for therapeutic applications.

### DETAILED DESCRIPTION OF THE INVENTION

During the course of a search for genes differentially expressed in human tumor cell lines established from primary and matched (from the same patient) metastatic head and neck squamous cell carcinoma (HNSCC), the present inventors identified a ˜2.0 kb transcript, corresponding to a partial cDNA clone SCC-S2, amplified in a metastatic and radioresistant HNSCC-derived cell line (PCI-06B) as compared to its matched primary tumor-derived cell line (PCI-06A) (17). Also of interest is the fact that PCI-06B cells are resistant to TNF-α-induced cytotoxicity (18).

As described in greater detail in the examples, studies were undertaken to isolate the full length SCC-S2 cDNA, determine the effect of TNF-α on SCC-S2 mRNA level in cancer cells, and to examine the possible anti-apoptotic function of SCC-S2. As shown infra, information obtained by the inventors suggests that SCC-S2 cDNA encodes a novel protein. The putative open reading frame (ORF) of SCC-S2 revealed significant homology with DED II of mouse and human FLIP proteins. Also, a GenBank database search has revealed that the SCC-S2 sequence reported here is similar to GG2-1 mRNA (accession number AF070671, ref #43) and MDC-3.13 isoform 1 mRNA (accession number AF099936). In addition, expressed sequence tags representing potential mouse and Drosophila homologues of human SCC-S2 cDNA were identified (accession numbers AA116718 and AA817594).

SCC-S2 mRNA is expressed in most human normal tissues and cancer cell lines. The inventors also confirmed their previous observation of a relatively higher steady state level of SCC-S2 mRNA in PCI-06B cells compared to PCI-06A cells, and demonstrated a significant TNF-α-inducible expression of SCC-S2 mRNA in different tumor cell types. In addition, transient expression of FLAG epitope-tagged SCC-S2 protein in HeLa cells was found to result in a decrease in the number of cells undergoing apoptosis in the presence or absence of TNF-α as compared to the vector transfectants.

Based on these discoveries, the present invention relates to a novel gene, SCC-S2, that negatively mediates apoptosis, the corresponding polypeptide, and application thereof in diagnostic and therapeutic methods. Particularly, the invention provides a novel target for identifying compounds that promote apoptosis of cancer cells, especially ovarium, renal, head and neck as well as some leukemias. With this general understanding, the invention is described in greater detail below.

As noted, the invention is broadly directed to a novel gene referred to as SCC-S2. Reference to SCC-S2 herein is intended to be construed to include SCC-S2 proteins of any origin which are substantially homologous to and which are biologically equivalent to the SCC-S2 characterized and described herein. Such substantially homologous SCC-S2s may be native to any tissue or species and, similarly, biological activity can be characterized in any of a number of biological assay systems.

The term “biologically equivalent” is intended to mean that the compositions of the present invention are capable of demonstrating some or all of the same biological properties in a similar fashion, not necessarily to the same degree as the SCC-S2 isolated as described herein or recombinantly produced human SCC-S2 of the invention.

By “substantially homologous” it is meant that the degree of homology of human SCC-S2 from any species is greater than that between SCC-S2 and any previously reported apoptopic modulating gene.

Sequence identity or percent identity is intended to mean the percentage of same residues between two sequences, wherein the two sequences are aligned using the Clustal method (Higgins et al, Cabios 8:189-191, 1992) of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise-alignment=5; diagonals saved in pairwise alignmentz=5. The residue weight table used for the alignment program is PAM250 (Dayhoff et al., in Atlas of Protein Sequence and Structure, Dayhoff, Ed., NDRF, Washington, Vol. 5, suppl. 3, p. 345, 1978).

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human SCC-S2 when determining percent conservation with non-human SCC-S2, and referenced to SCC-S2 when determining percent conservation with non-SCC-S2 proteins. Conservative amino acid changes satisfying this requirement are: R-K; E-D, Y-F, L-M; V-I, Q-H.

### Polypeptide Fragments

The invention provides polypeptide fragments of the disclosed proteins. Polypeptide fragments of the invention can comprise at least 8, 10, 12, 15, 18, 19, 20, 25, 50, 75, 100, 125, 130, 140, 150, 160, 170 or 180 contiguous amino acids of the amino acid sequence contained in FIG. 1 (SEQ ID NO: 2). Also included are all intermediate length fragments in this range, such as 101, 102, 103, etc.; 70, 71, 72, etc.; and 180, 181, 182, etc., which are exemplary only and not limiting.

### Biologically Active Variants

Variants of the SCC-S2 polypeptide disclosed herein can also occur. Variants can be naturally or non-naturally occurring. Naturally occurring variants are found in humans or other species and comprise amino acid sequences which are substantially identical to the amino acid sequence shown in FIG. 1 (SEQ ID NO: 2). Species homologs of the protein can be obtained using subgenomic polynucleotides of the invention, as described below, to make suitable probes or primers to screening cDNA expression libraries from other species, such as mice, monkeys, yeast, or bacteria, identifying cDNAs which encode homologs of the protein, and expressing the cDNAs as is known in the art.

Non-naturally occurring variants which retain substantially the same biological activities as naturally occurring protein variants are also included here. Preferably, naturally or non-naturally occurring variants have amino acid sequences which are at least 85%, 90%, or 95% identical to the amino acid sequence shown in FIG. 1 (SEQ ID NO: 2.). More preferably, the molecules are at least 96%, 97%, 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. (1981) 2:482-489.

Guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of SCC-S2 or polypeptide variants are of the same type as a protein comprising the amino acid sequence encoded by the nucleotide sequence shown in FIG. 1 (SEQ ID NO: 1), although the properties and functions of variants can differ in degree.

SCC-S2 protein variants include glycosylated forms, aggregative conjugates with other molecules, and covalent conjugates with unrelated chemical moieties. SCC-S2 protein variants also include allelic variants, species variants, and muteins. Truncations or deletions of regions which do not affect the differential expression of the SCC-S2 protein gene are also variants. Covalent variants can be prepared by linking functionalities to groups which are found in the amino acid chain or at the N- or C-terminal residue, as is known in the art.

It will be recognized in the art that some amino acid sequence of the SCC-S2 protein of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the SCC-S2 polypeptide which show comparable expression patterns or which include antigenic regions. Such mutants include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J Mol. Biol. 224:899-904 (1992) and de Vos et al. Science 255:306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

### Fusion Proteins

Fusion proteins comprising proteins or polypeptide fragments of SCC-S2 can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of SCC-S2 or a fragment thereof can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can utilize the amino acid sequence shown in FIG. 1 (SEQ ID NO: 2) or can be prepared from biologically active variants of FIG. 1 (SEQ ID NO: 2), such as those described above. The first protein segment can consist of a full-length SCC-S2.

Other first protein segments can consist of at least 8, 10, 12, 15, 18, 19, 20, 25, 50, 75, 100, 125, 130, 140, 150, 160, 170, 180 or 185 contiguous amino acids selected from SEQ ID NO: 2. The contiguous amino acids listed herein are not limiting and also include all intermediate lengths such as 20, 21, 22, etc.; 70, 71, 72, etc. and 180, 181, 182, 183, 184, 185, 186, 187, etc.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP 16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence contained in FIG. 1 (SEQ ID NO: 1) in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a coding sequence of the nucleotide sequence shown in FIG. 1 (SEQ ID NO: 1) can be expressed in prokaryotic or eukaryotic host cells using expression systems known in the art. These expression systems include bacterial, yeast, insect, and mammalian cells.

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein. Such covalent attachments can be made using known chemical or enzymatic methods.

SCC-S2 protein or polypeptide of the invention can also be expressed in cultured host cells in a form which will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides of the sequence contained in FIG. 1 (SEQ ID NO: 1) Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers from the nucleotide sequence shown in FIG. 1 (SEQ ID NO: 1) for use in identifying or amplifying the genes from mammalian, including human, genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

### Polynucleotide Constructs

Polynucleotide molecules comprising the coding sequences disclosed herein can be used in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

### Host Cells

An expression construct can be introduced into a host cell. The host cell comprising the expression construct can be any suitable prokaryotic or eukaryotic cell. Expression systems in bacteria include those described in Chang et al., Nature (1978) 275:615; Goeddel et al., Nature (1979) 281: 544; Goeddel et al., Nucleic Acids Res. (1980) 8:4057; EP 36,776; U.S. Pat. No. 4,551,433; deBoer et al., Proc. Natl. Acad Sci. USA (1983) 80: 21-25; and Siebenlist et al., Cell (1980) 20: 269.

Expression systems in yeast include those described in Hinnen et al., Proc. Natl. Acad. Sci. USA (1978) 75: 1929; Ito et al., J Bacteriol. (1983) 153: 163; Kurtz et al., Mol. Cell. Biol. (1986) 6:142; Kunze et al., J Basic Microbiol. (1985) 25:141; Gleeson et al., J. Gen. Microbiol. (1986) 132: 3459, Roggenkamp et al., Mol. Gen. Genet. (1986) 202:302); Das et al., J Bacteriol. (1984) 158: 1165; De Louvencourt et al., J Bacteriol. (1983) 154: 737, Van den Berg et al., Bio/Technology (1990) 8: 135; Kunze et al., J. Basic Microbiol. (1985) 25: 141; Cregg et al., Mol. Cell. Biol. (1985) 5: 3376; U.S. Pat. No. 4,837,148; U.S. Pat. No. 4,929,555; Beach and Nurse, Nature (1981) 300: 706; Davidow et al., Curr. Genet. (1985) 1p: 380; Gaillardin et al., Curr. Genet. (1985) 10: 49; Ballance et al., Biochem. Biophys. Res. Commun. (1983) 112: 284-289; Tilburn et al., Gene (1983) 26: 205-22; Yelton et al., Proc. Natl. Acad, Sci. USA (1984) 81: 1470-1474; Kelly and Hynes, EMBO J. (1985) 4: 475479; EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. (1988) 69: 765-776; Miller et al., Ann. Rev. Microbiol. (1988) 42: 177; Carbonell et al., Gene (1988) 73: 409; Maeda et al., Nature (1985) 315: 592-594; Lebacq-Verheyden et al., Mol. Cell Biol. (1988) δ: 3129; Smith et al., Proc. Natl. Acad. Sci. USA (1985) 82: 8404; Miyajima et al., Gene (1987) 58: 273; and Martin et al., DNA (1988) 7:99. Numerous baculoviral strains and variants and corresponding permissive insect host cells from hosts are described in Luckow et al., Bio/Technology (1988) δ: 47-55, Miller et al., in GENERIC ENGINEERING (Setlow, J. K. et al. eds.), Vol. 8 (Plenum Publishing, 1986), pp. 277-279; and Maeda et al., Nature, (1985) 315: 592-594.

Mammalian expression can be accomplished as described in Dijkema et al., EMBO J. (1985) 4: 761; Gorman et al., Proc. Natl. Acad. Sci. USA (1982b) 79: 6777; Boshart et al., Cell (1985) 41: 521; and U.S. Pat. No. 4,399,216. Other features of mammalian expression can be facilitated as described in Ham and Wallace, Meth Enz. (1979) 58: 44; Barnes and Sato, Anal. Biochem. (1980) 102: 255; U.S. Pat. No. 4,767,704; U.S. Pat. No. 4,657,866; U.S. Pat. No. 4,927,762; U.S. Pat. No. 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides from the nucleotide sequence shown in FIG. 1 (SEQ ID NO: 1). The transcription unit is located upstream to a coding sequence of the endogenous gene. The exogenous regulatory sequence directs transcription of the coding sequence of the endogenous gene.

SCC-S2 can also include hybrid and modified forms of SCC-S2 proteins including fusion proteins, SCC-S2 fragments and hybrid and modified forms in which certain amino acids have been deleted or replaced, modifications such as where one or more amino acids have been changed to a modified amino acid or unusual amino acid, and modifications such as glycosylations so long as the hybrid or modified form retains at least one of the biological activities of SCC-S2. By retaining the biological activity of SCC-S2, it is meant that the protein modulates cancer cell proliferation or apoptosis, although not necessarily at the same level of potency as that of SCC-S2 as described herein.

Also included within the meaning of substantially homologous is any SCC-S2 which may be isolated by virtue of cross-reactivity with antibodies to the SCC-S2 described herein or whose encoding nucleotide sequences including genomic DNA, mRNA or cDNA may be isolated through hybridization with the complementary sequence of genomic or subgenomic nucleotide sequences or cDNA of the SCC-S2 herein or fragments thereof. It will also be appreciated by one skilled in the art that degenerate DNA sequences can encode human SCC-S2 and these are also intended to be included within the present invention as are allelic variants of SCC-S2.

Preferred SCC-S2 of the present invention have been identified and isolated in purified form as described. Also preferred is SCC-S2 prepared by recombinant DNA technology. By “pure form” or “purified form” or “substantially purified form” it is meant that a SCC-S2 composition is substantially free of other proteins which are not SCC-S2.

The present invention also includes therapeutic or pharmaceutical compositions comprising SCC-S2 in an effective amount for treating patients with disease, and a method comprising administering a therapeutically effective amount of SCC-S2. These compositions and methods are useful for treating a number of diseases including cancer. One skilled in the art can readily use a variety of assays known in the art to determine whether SCC-S2 would be useful in promoting survival or functioning in a particular cell type.

In certain circumstances, it may be desirable to modulate or decrease the amount of SCC-S2 expressed. Thus, in another aspect of the present invention, SCC-S2 anti-sense oligonucleotides can be made and a method utilized for diminishing the level of expression of SCC-S2 by a cell comprising administering one or more SCC-S2 anti-sense oligonucleotides. By SCC-S2 anti-sense oligonucleotides reference is made to oligonucleotides that have a nucleotide sequence that interacts through base pairing with a specific complementary nucleic acid sequence involved in the expression of SCC-S2 such that the expression of SCC-S2 is reduced. Preferably, the specific nucleic acid sequence involved in the expression of SCC-S2 is a genomic DNA molecule or mRNA molecule that encodes SCC-S2. This genomic DNA molecule can comprise regulatory regions of the SCC-S2 gene, or the coding sequence for mature SCC-S2 protein.

The term complementary to a nucleotide sequence in the context of SCC-S2 antisense oligonucleotides and methods therefor means sufficiently complementary to such a sequence as to allow hybridization to that sequence in a cell, i.e., under physiological conditions. The SCC-S2 antisense oligonucleotides preferably comprise a sequence containing from about 8 to about 100 nucleotides and more preferably the SCC-S2 antisense oligonucleotides comprise from about 15 to about 30 nucleotides. The SCC-S2 antisense oligonucleotides can also contain a variety of modifications that confer resistance to nucleolytic degradation such as, for example, modified internucleoside linages (Uhlmann and Peyman, Chemical Reviews 90:543-548 1990; Schneider and Banner, Tetrahedron Lett. 31:335, 1990 which are incorporated by reference), modified nucleic acid bases as disclosed in 5,958,773 and patents disclosed therein, and/or sugars and the like.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art, a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth, proliferation or viability as is known in the art. Assays for measuring apoptosis are also known.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. 23:45-50, 1998.)

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

SCC-S2 can also be linked or conjugated with agents that provide desirable pharmaceutical or pharmacodynamic properties. For example, SCC-S2 can be coupled to any substance known in the art to promote penetration or transport across the blood-brain barrier such as an antibody to the transferrin receptor, and administered by intravenous injection (see, for example, Friden et al., Science 259:373-377, 1993 which is incorporated by reference). Furthermore, SCC-S2 can be stably linked to a polymer such as polyethylene glycol to obtain desirable properties of solubility, stability, half-life and other pharmaceutically advantageous properties. (See, for example, Davis et al., Enzyme Eng. 4:169-73, 1978; Buruham, Am. J. Hosp. Pharm. 51:210-218, 1994 which are incorporated by reference.)

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. SCC-S2 can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing SCC-S2 are to be administered orally. Such formulations are preferably encapsulated and formulated with suitable carriers in solid dosage forms. Some examples of suitable carriers, excipients, and dilutents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, SCC-S2 may be therapeutically administered by implanting into patients vectors or cells capable of producing a biologically-active form of SCC-S2 or a precursor of SCC-S2, i.e., a molecule that can be readily converted to a biological-active form of SCC-S2 by the body. In one approach cells that secrete SCC-S2 may be encapsulated into semipermeable membranes for implantation into a patient. The cells can be cells that normally express SCC-S2 or a precursor thereof or the cells can be transformed to express SCC-S2 or a precursor thereof. It is preferred that the cell be of human origin and that the SCC-S2 be human SCC-S2 when the patient is human. However, the formulations and methods herein can be used for veterinary as well as human applications and the term “patient” as used herein is intended to include human and veterinary patients.

In a number of circumstances it would be desirable to determine the levels of SCC-S2 in a patient. The identification of SCC-S2 along with the present report showing expression of SCC-S2 provides the basis for the conclusion that the presence of SCC-S2 serves a normal physiological function related to cell growth and survival. Endogenously produced SCC-S2 may also play a role in certain disease conditions.

The term “detection” as used herein in the context of detecting the presence of SCC-S2 in a patient is intended to include the determining of the amount of SCC-S2 or the ability to express an amount of SCC-S2 in a patient, the estimation of prognosis in terms of probable outcome of a disease and prospect for recovery, the monitoring of the SCC-S2 levels over a period of time as a measure of status of the condition, and the monitoring of SCC-S2 levels for determining a preferred therapeutic regimen for the patient.

To detect the presence of SCC-S2 in a patient, a sample is obtained from the patient. The sample can be a tissue biopsy sample or a sample of blood, plasma, serum, CSF or the like. SCC-S2 tissue expression is disclosed in the examples. Samples for detecting SCC-S2 can be taken from these tissue. When assessing peripheral levels of SCC-S2, it is preferred that the sample be a sample of blood, plasma or serum. When assessing the levels of SCC-S2 in the central nervous system a preferred sample is a sample obtained from cerebrospinal fluid or neural tissue.

In some instances it is desirable to determine whether the SCC-S2 gene is intact in the patient or in a tissue or cell line within the patient. By an intact SCC-S2 gene, it is meant that there are no alterations in the gene such as point mutations, deletions, insertions, chromosomal breakage, chromosomal rearrangements and the like wherein such alteration might alter production of SCC-S2 or alter its biological activity, stability or the like to lead to disease processes. Thus, in one embodiment of the present invention a method is provided for detecting and characterizing any alterations in the —SCC-S2 gene. The method comprises providing an oligonucleotide that contains the SCC-S2 cDNA, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize to the SCC-S2 gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, Taql and Alul. Using the Southern blot protocol, which is well known in the art, this assay determines whether a patient or a particular tissue in a patient has an intact SCC-S2 gene or a SCC-S2 gene abnormality.

Hybridization to a SCC-S2 gene would involve denaturing the chromosomal DNA to obtain a single-stranded DNA; contacting the single-stranded DNA with a gene probe associated with the SCC-S2 gene sequence; and identifying the hybridized DNA-probe to detect chromosomal DNA containing at least a portion of a human SCC-S2 gene.

The term “probe” as used herein refers to a structure comprised of a polynucleotide that forms a hybrid structure with a target sequence, due to complementarity of probe sequence with a sequence in the target region. Oligomers suitable for use as probes may contain a minimum of about 8-12 contiguous nucleotides which are complementary to the targeted sequence and preferably a minimum of about 20.

The SCC-S2 gene probes of the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32° 40° C. and more preferably at 37°-38° C. The time required for hybridization is from about 0.25 to about 96 hours, more preferably from about one to about 72 hours, and most preferably from about 4 to about 24 hours.

SCC-S2 gene abnormalities can also be detected by using the PCR method and primers that flank or lie within the SCC-S2 gene. The PCR method is well known in the art. Briefly, this method is performed using two oligonucleotide primers which are capable of hybridizing to the nucleic acid sequences flanking a target sequence that lies within a SCC-S2 gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising SCC-S2 or a fragment thereof is then directly sequenced and analyzed by comparison of the sequence with the sequences disclosed herein to identify alterations which might change activity or expression levels or the like.

In another embodiment, a method for detecting SCC-S2 is provided based upon an analysis of tissue expressing the SCC-S2 gene. Certain tissues such as those identified below in Example 6 and 7 have been found to express the SCC-S2 gene. The method comprises hybridizing a polynucleotide to mRNA from a sample of tissue that normally expresses the SCC-S2 gene. The sample is obtained from a patient suspected of having an abnormality in the SCC-S2 gene or in the SCC-S2 gene of particular cells.

To detect the presence of mRNA encoding SCC-S2 protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of a labeled probes as discussed above allows detection of the resulting duplex.

When using the cDNA encoding SCC-S2 protein or a derivative of the cDNA as a probe, high stringency conditions can be used in order to prevent false positives, that is the hybridization and apparent detection of SCC-S2 nucleotide sequences when in fact an intact and functioning SCC-S2 gene is not present. When using sequences derived from the SCC-S2 cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. (Sambrook et al., 1989, supra).

In order to increase the sensitivity of the detection in a sample of mRNA encoding the SCC-S2 protein, the technique of reverse transcription/polymerization chain reaction (RT/PCR) can be used to amplify cDNA transcribed from mRNA encoding the SCC-S2 protein. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using α-reverse transcriptase enzyme and a 3′ end primer. Typically, the primer contains an oligo(dT) sequence. The cDNA thus produced is then amplified using the PCR method and SCC-S2 specific primers. (Belyavsky et al., Nucl. Acid Res. 17:2919-2932, 1989; Krug and Berger, Methods in Enzymology, 152:316-325, Academic Press, NY, 1987 which are incorporated by reference).

The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence of the SCC-S2 protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. (Basic and Clinical Immunology, 217-262, Sites and Terr, eds., Appleton & Lange, Norwalk, Conn., 1991, which is incorporated by reference). Preferred are binder-ligand immunoassay methods including reacting antibodies with an epitope or epitopes of the SCC-S2 protein and competitively displacing a labeled SCC-S2 protein or derivative thereof.

As used herein, a derivative of the SCC-S2 protein is intended to include a polypeptide in which certain amino acids have been deleted or replaced or changed to modified or unusual amino acids wherein the SCC-S2 derivative is biologically equivalent to SCC-S2 and wherein the polypeptide derivative cross-reacts with antibodies raised against the SCC-S2 protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

Polyclonal or monoclonal antibodies to the protein or an epitope thereof can be made for use in immunoassays by any of a number of methods known in the art. By epitope reference is made to an antigenic determinant of a polypeptide. An epitope could comprise 3 amino acids in a spatial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, usually a rabbit or a mouse.

Oligopeptides can be selected as candidates for the production of an antibody to the SCC-S2 protein based upon the oligopeptides lying in hydrophilic regions, which are thus likely to be exposed in the mature protein. Peptide sequence used to generate antibodies against any fragment of SCC-S2 that typically is at least 5-6 amino acids in length, optionally fused to an immunogenic carrier protein, e.g. KLH or BSA.

Additional oligopeptides can be determined using, for example, the Antigenicity Index, Welling, G. W. et al., FEBS Lett. 188:215-218 (1985), incorporated herein by reference.

In other embodiments of the present invention, humanized monoclonal antibodies are provided, wherein the antibodies are specific for SCC-S2. The phrase “humanized antibody” refers to an antibody derived from a non-human antibody, typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321:522-525 (1986); Morrison et al., Proc. Natl. Acad. Sci, U.S.A., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human-heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell receptors. See, e.g., U.S. Pat. Nos. 5,530,101 and 5,585,089 which patents are incorporated herein by reference.

Humanized antibodies to SCC-S2 can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNF, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, SCC-S2 polypeptides of the invention and variants thereof are used to immunize a transgenic animal as described above. Monoclonal antibodies are made using methods known in the art, and the specificity of the antibodies is tested using isolated SCC-S2 polypeptides.

Methods for preparation of the SCC-S2 protein or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system (E. I. du Pont de Nemours Company, Wilmington, Del.) (Caprino and Han, J. Org. Chem. 37:3404, 1972 which is incorporated by reference).

Polyclonal antibodies can be prepared by immunizing rabbits or other animals by injecting antigen followed by subsequent boosts at appropriate intervals. The animals are bled and sera assayed against purified SCC-S2 protein usually by ELISA or by bioassay based upon the ability to block the action of SCC-S2. In a non-limiting example, an antibody to SCC-S2 can block the binding of SCC-S2 to Disheveled protein. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the SCC-S2 protein by treatment of a patient with specific antibodies to the SCC-S2 protein.

Specific antibodies, either polyclonal or monoclonal, to the SCC-S2 protein can be produced by any suitable method known in the art as discussed above. For example, murine or human monoclonal antibodies can be produced by hybridoma technology or, alternatively, the SCC-S2 protein, or an immunologically active fragment thereof, or an anti-idiotypic antibody, or fragment thereof can be administered to an animal to elicit the production of antibodies capable of recognizing and binding to the SCC-S2 protein. Such antibodies can be from any class of antibodies including, but not limited to IgG, IgA, 1gM, IgD, and IgE or in the case of avian species, IgY and from any subclass of antibodies.

The availability of SCC-S2 allows for the identification of small molecules and low molecular weight compounds that inhibit the binding of SCC-S2 to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds may be identified via the incorporation of radioactivity or through optical assays that rely on absorbence, fluorescence or luminescence as read-outs. Gonzalez, J. E. et al., (1998) Curr. Opin. Biotech. 9:624-63 1.

Model systems are available that can be adapted for use in high throughput screening for compounds that inhibit the interaction of SCC-S2 with its ligand, for example by competing with SCC-S2 for ligand binding. Sarubbi et al., (1996) Anal. Biochem. 237:70-75 describe cell-free, non-isotopic assays for discovering molecules that compete with natural ligands for binding to the active site of IL-1 receptor. Martens, C. et al., (1999) Anal. Biochem. 273:20-31 describe a generic particle-based nonradioactive method in which a labeled ligand binds to its receptor immobilized on a particle; label on the particle decreases in the presence of a molecule that competes with the labeled ligand for receptor binding.

The therapeutic SCC-S2 polynucleotides and polypeptides of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. 166:154-165 (1988); and Flotte et. al., P.N.A.S. 90:10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Bisleret al., P.N.A.S. 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5:1287-1291 (1993); Vincent et al., Nat. Genet. 5:130-134 (1993); Jaffe et al., Nat. Genet. 1:372-378 (1992); and Levrero et al., Gene 101:195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3:147-154 (1992) may be employed.

Other gene delivery vehicles and methods may be employed, including polycationic condensed DNA linked or unlinked to killed adenovirus alone, for example Curiel, Hum. Gene Ther. 3:147-154 (1992); ligand-linked DNA, for example see Wu, J. Biol. Chem. 264:16985-16987 (1989); eukaryotic cell delivery vehicles cells; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13796, WO 94/23697, and WO 91/14445, and EP No. 0-524 968.

Further non-viral delivery suitable for use includes mechanical delivery systems such as the approach described in Woffendin et al., Proc. Natl. Acad. Sci. USA 91(24):11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

SCC-S2 may also be used in screens to identify drugs for treatment of cancers which involve over-activity of the encoded protein, or new targets which would be useful in the identification of new drugs.

For all of the preceding embodiments, the clinician will determine, based on the specific condition, whether SCC-S2 polypeptides or polynucleotides, antibodies to SCC-S2, or small molecules such as peptide analogues or antagonists, will be the most suitable form of treatment. These forms are all within the scope of the invention.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary of the scope and spirit of the invention.

### EXAMPLES

### Example 1

### Identification, Sequencing, Cloning and Expressing and Functional Assay for SCC-S2 in Transferred Cells

The following procedures and materials were used in order to identify, sequence and clone SCC-S2 cDNA from human cancer cell lines that overexpress this protein:

### Cell Culture

HNSCC cell lines, PCI-06A, PCI-06B, and PCI-04A (19) were grown in minimal essential medium (MEM) supplemented with 15% heat-inactivated fetal bovine serum (FBS), 10 mM HEPES buffer, 1 mM non-essential amino acids, 2 mM L-glutamine, 25 μg/ml gentamicin, all from GIBCO-BRL and 0.4 μg/ml hydrocortisone (Sigma). The other human tumor cell lines were grown in Improved MEM (Celigro) containing 10% heat-inactivated FBS. The cells were grown in 75 cm² tissue culture flasks in a humidified atmosphere of 5% CO₂, and 95% air at 37° C.

cDNA Cloning

A human heart cDNA library in λZapII-vector (Stratagene) was screened using a ³²P-labeled SCC-S2 partial cDNA fragment as probe (17). In brief, ˜1×10⁶ Plaque forming units were screened. The filters were hybridized at 42° C. in buffer containing 50% formamide, 5×SSC, 1×Denhardt's solution, 20 mM sodium phosphate buffer (pH 6.8), and 200 μg/ml sheared salmon sperm DNA, followed by washings at 55° C., three times in 2×SSC and 0.1% SDS, and three times in 0.2×SSC and 0.1% SDS. The filters were rinsed twice in 2×SSC, damp dried and autoradiographed. The positive clones were isolated after five cycles of amplification and screening. The cDNA insert (1519 bp) from a positive clone (ID#DK721) was subcloned into pBluescript (+) vector by in vivo excision according to the manufacturer's instructions (Stratagene).

### Sequence Analysis and Database Search

Both strands of the SCC-S2 cDNA (1519 bp) were sequenced by automated sequencing using Applied Biosystems Prism 377 DNA sequencer and an Applied Biosystems, Prism Dye terminator cycle reaction kit (Perkin Elmer). Raw data files from ABI 377 sequencer were imported into Auto Assembler program (ABI). Contigs were generated by comparing all fragments in one project with the parameters of at least 50 bp overlap and at least 75% level of homology. The assembled sequence was used to find a matching I.M.A.G.E. consortium EST clone AA 406630 from human EST database (20). The I.M.A.G.E. EST clone AA 406630 was purchased from Genome Systems and sequenced as above. The sequences were assembled using the Auto Assembler program, and the complete sequence was then subjected to database search. Sequence database search and ORF prediction were done using the National Center for Biotechnological Information (NCBI) BLAST and ORF finder programs on world wide web at. http://www.ncbi.nlm.nih.gov (21). Multiple sequence alignment was performed using MultiAlign program at http://www.toulouse.inra.fr/multalin.html (22). The search for the presence of different motifs and signature sequences was conducted at http://www.motif.genome.ad.ip/motif-bin/nph-motif2. The prediction for the possible nature of putative protein based on structural characteristics was done by Reinhardt's method at http://Psort.nibb.ac.ip:8800/cqi-bin/runPsort.pl (23).

### TNF-α Treatment, Northern Blotting and Hybridization

Logarithmically growing cells were switched to serum free medium for 2 h prior to the addition of the indicated amounts of TNF-α (R & D Systems), followed by incubations for various times as described before (24, 25). The cells were washed with cold PBS and total RNA was isolated with Trizol reagent according to the manufacturer's specifications (GIBCO/BRL). For northern analysis, total RNA was electrophoresed on 1% agarose-formaldehyde gel, transferred overnight to nylon membrane (Qiagen), fixed by UV crosslinking, and membrane was baked at 80° C. for 2 h. The multi-tissue blots H, H2, H3, F and C blots containing poly A+ RNA from adult and fetal tissues and various cancer cell lines were purchased from Clontech. 10⁶ cpm/ml of ³²P-labeled SCC-S2 cDNA (˜1.5 kb, ID # DK721) was used as probe, and hybridizations were performed at 68° C. using ExpressHyb (Clontech), followed by washings with 2×SSC and 0.1% SDS at room temperature and 0.1×SSC containing 0.1% SDS at 68° C. as described before (17, 26). Membranes were reprobed with radiolabeled glyceraldehyde-3-phosphate dehydrogenase (GAPDH) or β-actin probe as an internal control. The autoradiographs were scanned and bands quantified using ImageQuant software version 3.3 (Molecular Dynamics Personal Densitometer).

PCR Amplification and Cloning of FLAG Epitope-Tagged SCC-S2 cDNA in Mammalian Expression Vector

cDNA fragment encoding the open reading frame of SCC-S2 (nucleotides 1-697, FIG. 1) was amplified by PCR using human placental cDNA (Clontech). The 5′- and 3′-primers used for amplification were 5′-CCCAAGCTTCTCCCGCCGGCTCT AACC-3′ (SEQ ID NO:23) and 5′ CCAGGAATTCTCA CTT GTC ATC GTC GTC CTT GTA GTC TATGTTCTCT TCATCCAAC-3′ (SEQ ID NO:24), respectively. The sequence underlined in the 3′ primer corresponds to the FLAG octapeptide (Sigma). The amplified product (734 bp) was verified by automated sequence analysis of both strands, and cloned into the mammalian expression vector PCR 3.1 according to the instruction manual (Invitrogen).

### Transient Transfection and Immunoblottinq

HeLa cells were seeded in six well plates (1−2×10⁵ cells/well) and transfected with the expression vector PCR 3.1 or recombinant vector containing FLAG-tagged SCC-S2 cDNA (2 μg/well) using the LipofectAMINE™ method (Life Technologies, Inc.). 36 h after transfection, cells were harvested and lysed at 4° C. for 30 min in lysis buffer (100 mM HEPES, pH 7.5, 1% NP-40, 150 mM NaCl, 10% Glycerol, 1 mM PMSF, and 10 μg/ml each of aprotinin and leupeptin), followed by microcentri-fugation for 5 min at 4° C. Protein concentration was determined using Coomasie G250 protein assay reagent (Pierce). Cell lysates (25-50 μg) were resolved by 15% SDS-PAGE, transferred to an Immobilon-P membrane (Millipore), and immunoblotted with 1 μg/ml of the mouse monoclonal FLAG-M2 antibody (Sigma). Enhanced chemiluminescence method (Luminol, NEN) was used to detect the signal. Blot was reprobed with human polyclonal anti-GAPDH antibody (Trevigen)

### Apoptosis Assay

Hela cells were transiently transfected with vector or FLAG epitope-tagged SCC-S2 cDNA as described above. 30 h after transfection, cells were switched to medium containing 1% FBS for 1 h, and then treated with TNF-α (100 ng/ml) for additional 4 h. After treatment, floating cells were pooled with the adherent cells collected by trypsinization, and fixed in 2 ml of 75% ethanol for at least 30 min at 4° C. For the FACS analysis of sub-G1 cells, the fixed cells were pelleted and resuspended in 1 ml of phosphate-buffered saline solution containing 50 μg/ml each of RNase A (Sigma) and propidium iodide (Sigma). The stained cells were analyzed using a FACsort (Becton-Dickinson), and Reproman computer software. The percentage of cells containing sub-G1 DNA content was used as an index of apoptosis as described (27, 28).

### Example 2

### SCC-S2 mRNA is Overexpressed in Primary or Metastatic Tumor Specimens

Matched sets of tumor and normal adjacent tissue specimens were procured through the Co-operative Human Tissue Network resource of the National Cancer Institute (NIH). Portions of these specimens were processed for histopathological analysis, and the remainder of the samples were used for SCC-S2 mRNA expression analysis (FIG. 7). Densitometer scanning of the RNA blots indicated a 2.8- and 3.4-fold increase in the expression level of SCC-S2 mRNA in two primary renal cell carcinoma specimens, 2P-R and 1P-R, respectively, as compared to matched normal adjacent tissues (2N-R and 1N-R). In a third patient with ovarian carcinoma, a 2-fold increase in SCC-S2 expression was seen in a metastatic tumor (3M-OV) as compared to matched normal adjacent tissue (3N-OV). These data support the hypothesis that SCC-S2 plays a role in tumor progression.

### Example 3

### SCC-S2 mRNA is Induced by Androgen, R1881

Hormone-responsive LnCap prostate cancer cells were grown in IMEM with 5% FBS. Cells were switched to medium containing 5% charcol-stripped serum for 24 h, and then indicated concentration of synthetic androgen, R1881 (NEN) was added for 48 h. Total RNA was analyzed by northern blotting using SCC-112 cDNA as probe as shown below (FIG. 8).

### Example 4

### SCC-S2 Expression Enhances Tumor Growth

SCC-S2 cDNA was cloned into a eukaryotic expression vector (FIG. 9, top left). Hormone-independent MDA-MB 435 human breast cancer cells were stably transfected with expression vector (PCR 3.1, EV) or vector containing Flag-tagged SCC-S2 cDNA (SCC-S2). The expression of exogenous SCC-S2 protein was detected in cell lysates by immunoblotting using anti-FLAG antibody (Sigma) (FIG. 9, top right). Female Balb/c athymic mice were injected s.c. in mammary fat pads with logarithmically growing 0.5×10⁶ MDA-MB 435 SCC-S2 transfectants or vector transfectants (n=5). The tumor sizes were measured at various times post-inoculation and tumor volumes were determined from caliper measurements of the three major axes (a,b,c) and calculated using abc/2, an approximation for the volume of an ellipse (IIabc/6). (FIG. 3, bottom left). Expression of exogenous SCC-S2 protein was detected in extracts of tumor tissues by immunoblotting with anti-FLAG antibody. Our data indicate that SCC-S2 expression potentiates tumor growth of this highly aggressive hormone-independent and metastatic breast tumor model.

### Example 5

### SCC-S2 Peptide Design, Antibody Production, and Testing

Based on the ORF (JBC, 2000), we have designed a peptide representing 76-91 aa of SCC-S2 (CYRNNQFNQDELALMEK (SEQ ID NO:25)). A rabbit polyclonal antibody against SCC-S2 synthetic peptide has been custom made (Zymed laboratories). Whole cell lysates of LnCap cells were treated with 1 nM synthetic androgen R1881 (DuPont) for 48 h, and proteins were resolved by SDS PAGE, followed by immunoblotting with SCC-S2 atisera. A 21 kDA human SCC-S2 protein was detected in untreated cells and found to be induced in the presence of androgen. These data suggest that androgen-induced SCC-S2 mRNA expression (FIG. 2) correlates with the enhanced level of protein.

### Experimental Results and Conclusions

We report here the isolation and characterization of a novel TNF-α-inducible gene, SCC-S2. Based on the nucleotide sequence, SCC-S2 open reading frame (ORF) contains a sequence in the amino-terminus which shows a significant homology to death-effector-domain (DED) II of cell death regulatory protein, FLICE-inhibitory protein (FLIP). Unlike FLIP, SCC-S2 ORF contains only one DED and lacks the carboxy-terminus caspase-like homology domain, raising the possibility that SCC-S2 may be a novel member of the FLIP family. SCC-S2 mRNA expression is found in most normal tissues and malignant cells. The steady state level of SCC-S2 mRNA is significantly induced by TNF-α in different tumor cells (TNF-α, 20 ng/ml, 3 h: A549, ˜2-9 fold; SKOV-3, ˜3 fold; PCI-04A, ˜3-6 fold). TNF-α treatment (100 ng/ml, 4 h) of HeLa cells transiently transfected with FLAG epitope-tagged SCC-S2 cDNA or expression vector alone led to an increase in the number of apoptotic cells as compared to the untreated counterpart. Interestingly, however, SCC-S2 transfectants revealed a significant decrease in the number of apoptotic cells as compared to the vector transfectants (p<0.001). These data implicate a role of SCC-S2 as a negative mediator of apoptosis in certain cell types.

The screening of a human heart cDNA library with a partial SCC-S2 cDNA probe (259 bp, ref #17) led to the identification of a clone containing 1519 bp cDNA insert (ID#DK721). BLAST search of the EST database with this sequence resulted in the identification of a 5′-overlapping EST clone (AA 406630). The assembled nucleotide sequence was 1915 bp with a predicted ORF of 188 amino acids and an inframe stop codon 5′ to the first ATG (FIG. 1). The sequence contained 133 bp of the 5′- and 1215 bp of the 3′-untranslated regions. The polyadenylation signal sequence could be located in the 3′-untranslated region. SCC-S2 cDNA encoded for a putative cytosolic protein with predicted relative molecular mass of 21 kDa. Search for the known motifs and protein family signature sequences revealed three putative Casein Kinase II phosphorylation sites, and one Protein Kinase C phosphorylation site (FIG. 1).

BLAST search of the ORF indicate that SCC-S2 is a novel protein. The sequence contained a putative DED which showed significant homology with DED II of the FLIP family of cell death regulatory proteins. The putative DED domain in SCC-S2 showed identities (similarities) as follows: mouse CASH α/β, 35% (58%); human CASH α/β, 27% (50%); mouse FLIP(L), 32% (53%); and human FLIP(L), 27% (58%) (FIGS. 1 and 2). Identity higher than 25% is considered significant (29). The DDs and/or DEDs are important protein-protein interaction domains in death receptors including TNFR1, and adaptor molecules such as TRADD, FADD, FLICE, and RIP (receptor-interacting protein). Based on the known structure-functional relationships of FLIP proteins, presence of a putative DED domain in the N-terminus and absence of a caspase catalytic domain in the C-terminus suggest that SCC-S2 may serve as a dominant negative inhibitor of the DED containing molecules such as FLICE. Interestingly, the SCC-S2 DED shared only 9% and 11% identity with DED in mouse FLICE and human FLICE (32% and 38% similarity), respectively (FIG. 2). It is not known as yet whether SCC-S2 interacts with and/or inhibits FLICE.

Viral genomes are known to code for apoptosis inhibitory proteins, allowing increased viral replication to combat the host's apoptotic defense mechanism (5, 30-36). These inhibitors interact with Fas, TNF-receptor-related apoptosis-mediated protein (TRAMP), TNF-related apoptosis-inducing ligand receptor (TRAIL-R), and TNFR1, and block apoptotic signaling events. The poxvirus encoded serpin CrmA and baculovirus gene product p35 exert inhibitory effects by binding directly to FLICE (36). The putative SCC-S2 DED showed significant homology to the corresponding domains present in some viral proteins, sharing 30% and 46% identity (58% and 66% similarity) to human poliovirus coat proteins and canine adenovirus DNA polymerase, respectively (FIG. 2). Relatively weak identity (21%) and similarity (54%) of the SCC-S2 DED to vaccinia virus DNA polymerase were observed (FIG. 2). Other features of the SCC-S2 ORF included the signature sequence for vinculin family talin binding region proteins (FIG. 2). This sequence indicated 20% identity (44% similarity) to human α1(E)- and α2(E)-catenins, a class of proteins known to play a role in epithelial cell-cell contacts (37).

SCC-S2 transcript (˜2.0 kb) was detectable in most human normal tissues, with relatively higher levels in spleen, lymph node, thymus, thyroid, bone marrow and placenta, and lower levels in spinal cord, ovary, lung, adrenal glands, heart, brain, testis, and skeletal muscle (FIG. 3). Among the fetal tissues examined, a prominent signal was seen in liver, lung and kidney, whereas expression could not be detected in brain (FIG. 3). SCC-S2 mRNA was expressed in all cancer cell lines tested with relatively higher levels in K562 chronic myelogenous leukemia cells, MOLT 4 lymphoblastic leukemia cells, and A549 lung carcinoma cells, and lower in SW480 colorectal adenocarcinoma cells (FIG. 4). Consistent with our original findings (17), a 2.0 kb transcript was detected in PCI-06B cells, and SCC-S2 mRNA expression was reproducibly higher in PCI-06B cells than in PCI-06A cells (>2 fold) (FIG. 4).

Engagement of TNFR1 by its cognate ligand leads to increased expression of a number of pro- and anti-apoptotic genes. We asked whether TNF-α treatment of cells results in the induction of SCC-S2 mRNA. Data shown in FIG. 5 indicate a significant increase in the steady state level of SCC-S2 mRNA in A549 lung carcinoma cells, SKOV-3 ovarian carcinoma cells, and PCI-04A HNSCC cells (TNF-α, 20 ng/ml, 3 h: A549, ˜2-9 fold; SKOV-3, ˜3 fold; PCI-04A, ˜3-6 fold). It should be noted that A549 cells and SKOV-3 cells are resistant to TNF-α (38,39). TNF-α-induced SCC-S2 mRNA was also noted in U373MG cells and human hepatoma HepG2 cells (data not shown). TNF-α-inducible gene expression has been associated with the presence of binding motifs of transcription factors NF-κB and AP-1 in the promoter region of several genes. Whether SCC-S2 promoter contains a TNF-α-responsive element(s) remains to be determined.

To address the possibility of an anti-apoptotic function of SCC-S2, HeLa cells were transiently transfected with FLAG epitope-tagged SCC-S2 cDNA expression vector (FIG. 6, Left panel). The efficiency of transient transfection was initially determined by co-transfection with pCMV β-galactosidase expression vector (Clontech), and the percentage of blue cells was reproducibly comparable in vector and SCC-S2 transfectants (data not shown). The increase in number of cells in sub-G1 phase has been used as an indicator of apoptosis (40). Our data shows that TNF-α treatment of vector or SCC-S2 transfectants led to an increase in the number of cells in sub-G1 as compared to the untreated counterpart (FIG. 6, Right panel). Interestingly, however, expression of exogenous SCC-S2 resulted in a significant decrease in number of cells in sub-G1 phase, in the presence or absence of TNF-α, as compared to vector transfectants (p<0.001). These data suggest that SCC-S2 overexpression per se is a negative mediator of apoptosis.

The molecular genetic factors that negate cell death and contribute to tumor progression can be attractive targets for therapeutic intervention (41, 42). This supports the potential role of SCC-S2 in cancer progression, and the use thereof for design of novel cancer therapy and prophylaxis. The fact that SCC-S2 expression apparently inhibits apoptosis of cancer cells suggests that SCC-S2 has application in the design of novel antisense and ribozymal cancer therapies and for the identification of small molecules and antibodies that modulate the expression of SCC-S2 in cancers wherein tumor growth and/or metastatis is affected by SCC-S2 expression.

The following list of references are cited herein and are incorporated by reference in their entirety herein.

The present invention has been described with reference to specific embodiments. However, this invention is intended to cover those changes and substitutions, which may be made by those skilled in the art without departing from the spirit and scope of the appended claims.

### REFERENCES

1. Ashkenazi, A., and Dixit, V. M. (1998) Science 281, 1305-1308
2. Chinnaiyan, A. M., Tepper, C. G., Seldin, M. F., O'Rourke, K., Kischkel, F. C., Hellbardt, S., Krammer, P. H., Peter, M. E. and Dixit, V. M. (1996) J. Biol. Chem. 271, 4961-4965
3. Boldin, M. P., Goncharov, T. M., Goltsev, Y. V., and Wallach, D. (1996) Cell 85, 803-815
4. Muzio, M., Chinnaiyan, A. M., Kischkel, F. C., O'Rourke, K., Shevchenko, A., Ni, J., Scaffidi, C., Bretz, J. D., Zhang, M., Gentz, R., Mann, M., Krammer, P. H., Peter, M. E., and Dixit, V. M. (1996) Cell 85, 817-827
5. Thome, M., Schneider, P., Hofmann, K., Fickenscher, H., Meini, E., Neipel, F., Mattmann, C., Burns, K., Bodmer, J. L., Schroter, M., Scaffidi, C., Krammer, P. H., Peter, M. E., and Tschopp, J. (1997) Nature 386, 517-521
6. Srinivasula, S. M., Ahmad, M., Ottilie, S., Bulirich, F., Banks, S., Wang, Y., Fernandes-Alnemri, T., Croce, C. M., Litwack, G., Tomaselli, K. J., Armstrong, R. C., and Alnemri, E. S. (1997) (1997) J. Biol. Chem. 272, 18542-18545
7. Hu, S., Vincenz, C., Ni, J., Gentz, R., and Dixit, V. M. (1997) J. Biol. Chem. 272, 17255-17257
8. Irmier, M., Thome, M., Hahne, M., Schneider, P., Hofmann, K., Steiner, V., Bodmer, J. L., Schroter, M., Burns, K., Mattmann, C., Rimoldl, D., French, L. E., and Tschopp, J. (1997) Nature 388, 190-195
9. Goltsev, Y. V., Kovalenko, A. V., Arnold, E., Varfolomeev, E. E., Brodianskii, V. M., and Wallach, D. (1997) J. Biol. Chem. 272, 19641-19644
10. Griffith, T. S., Chin, W. A., Jackson, G. C., Lynch, D. H., and Kubin, M. Z. (1998) J. Immunol. 161, 2833-2840
11. Kataoka, T., Schroter, M., Hahne, M., Schneider, P., Immler, M., Thome, M., Foelich, C. J., and Tschopp, J. (1998) J. Immunol. 161, 3936-3942
12. Sata, M., and Walsh, K. (1998) J. Biol. Chem. 273, 33103-33106
13. Inbal, B., Cohen, O., Polak-Charcon, S., Kopolovic, J., Vadai, E., Eisenbach, L., and Kimchi, A. (1997) Nature 390, 180
14. Troppmair, J., Cleveland, J. L., Askew, D. S., and Rapp, U. R. (1992) Curr. Top. Microbiol. Immunol. 182, 453-460
15. Kasid, U. and Suy, S. (1998) In: Apoptosis Genes, eds. Potten, C. S., Booth, C., and Wilson, J. (Kluwer Academic Publishers, MA), p 85-118
16. Yeh, J. H., Hsu, S. C., Han, S. H., and Lai, M. Z. (1998) J. Exp. Med. 188(10), 1795-1802
17. Patel, S., Wang, F.-H., Whiteside, T. L., and Kasid, U. (1997) Oral Oncology, Eur. J. Cancer 33(3), 197-203
18. Sacchi, M., Klapan, I., Johnson, J. T., and Whiteside, T. L. (1991) Arch. Otolaryngol. Head Neck Surg. 117, 321-326
19. Heo, D. S., Snyderman, C., Gollin, S. M., Pan, S., Walker, E., Deka, R., Barnes, E. L., Johnson, J. T., Herberman, R. B., and Whiteside, T. L. (1989) Cancer Res 49:5167-5175
20. Lennon, G., Auffray, C., Polymeropoulos. M., Soares, M. B. (1996) Genomics 33(1), 151-152
21. Altschul, S. F., Madden, T. L., Schaffer, A. A., Zhang, J., Zhang, Z., Miller, W., and Lipman, D. J. (1997) Nucleic. Acids Res. 25, 3389-3402
22. Corpet, F. (1988) Nucleic Acids Res. 16, 10881-10890
23. Nakai, K., and Kanehisa, M. (1992) Genomics 14, 897-911
24. Kasid, U., Wang, F-H., and Whiteside, T. L. (1997) Mol. Cell. Biochem. 173, 193-197
25. Patel, S., Wang, F-H., Whiteside, T. L., and Kasid, U. (1998) Acta Oncologica 37, 475-478
26. Patel, S., Wang, F-H., Whiteside, T. L., and Kasid, U. (1997) Molecular Medicine 3, 674-685
27. Nicoletti, I., Migliorati, G., Pagliacci, M. C., Grignani, F., and Riccardi, C. (1991) J. Immuno. Methods 139, 271-279
28. Darzynkiewicz, Z., Bruno, S., Del Bino, G., Gorczyca, W., Hotz, M. A., Lassota, P., and Traganos, F. (1992) Cytometry 13, 795-808
29. Blundel, T. L., Sibanda, B. L., Sternberg, M. J. E., and Thornton, J. M. (1987) Nature 326, 347-352
30. Hu, S., Vincenz, C., Buller, M., and Dixit, V. M. (1997) J. Biol. Chem. 272, 9621-9624
31. Chio, S-K., and White, E. (1998) Virology 244, 108-118
32. Bertin, J. J., Armstrong, R. C., Ottilie, S., Martin, D., Wang, Y., Banks, S., Wabg, G-H., Senkevich, T. G., Alnemri, E. S., Moss, B., Lenardo, M. J., Tomaselli, K. J., and Cohen, J. I. (1997) Proc. Natl. Acad. Sci. USA 94, 1172
33. Earl, P. L., Jones, E. V., and Moss, B. (1986) Proc. Natl. Acad. Sci. USA 83, 3659-3663
34. Weiss, R. A., Whitby, D., Talbot, S., Kellam, P., and Boshoff, C. (1998) J. Natl. Cancer Inst. Mono. No. 23, 51-54
35. Hu, M. C., and Hsu, M. T. (1997) Virology 227, 295-304
36. Tewari, M., Telford, W. G., Miller, R. A. and Dixit, V. M. (1995) J. Biol. Chem. 270, 22705-22708
37. Oda, T., Kanai, Y., Shimoyama, Y., Nagafuchi, A., Tsukita, S., and Hirohashi, S. (1993) Biochem. Biophy. Res. Comm. 193(3), 897-904
38. Han, S. K., Brody, S. L., and Crystal, R. G. (1994) Am. J. Respir. Cell Mol. Biol. 11, 270-278
39. Morimoto, H., Safrit, J. T., and Bonavida, B. (1991) J. Biol. Chem. 147,
40. Kissil, J. L., Cohen, O., Raveh, T., and Kimchi, A. (1999) The EMBO J. 18,
41. Kasid, U., Pfeifer, A. M., Brennan, T., Beckett, M., Weichselbaum, R. R., Dritschilo, A., and Mark, G. E. (1989) Science 243, 1354-1356
42. Gokhale, P., McRae, D., Brett, M. P., Bagg, A., Rahman, A., Dritschilo, A., and Kasid, U. (1999) Antisense and Nucleic Acid Drug Development 9,
43. Horrevoets, A. J. G., Fontijn, R. D., Zonneveld, A. J. V., De Vries, C. J. M., Cate, J. W. T., and Pannekoek, H. (1999) Blood 93, 3418-3431

1.-42. (canceled)

43. A method of inhibiting proliferation of a cancer cell, comprising
a) contacting a cancer cell with a molecule that inhibits the expression of an SCC-S2 nucleic acid molecule, wherein the nucleic acid molecule is selected from the group consisting of:
(i) a nucleic acid molecule comprising SEQ ID NO: 1;
(ii) a nucleic acid molecule comprising nucleotides 397 to 1915 of SEQ ID NO: 1;
(iii) a nucleic acid molecule encoding a polypeptide comprising SEQ ID NO: 2;
(iv) a nucleic acid molecule encoding a polypeptide comprising an amino acid sequence having at least 85% sequence identity to SEQ ID NO: 2;
(v) a nucleic acid encoding a polypeptide comprising a functional fragment of SEQ ID NO: 2;
(vi) a nucleic acid molecule that hybridizes to (i)-(v) under stringent conditions; and
(vii) a nucleic acid molecule comprising a nucleic acid sequence fully complementary to (i)-(vi).
b) inhibiting expression of the SCC-S2 nucleic acid molecule in the cancer cell; and
c) inhibiting proliferation of the cancer cell.

44. The method of claim 43, wherein the molecule is selected from the group consisting of ribozyme and anti-sense oligonucleotide

45. The method of claim 43, wherein the cancer cell expresses higher levels of SCC-S2 mRNA than an equivalent non-cancer cell.

46. The method of claim 43, wherein the cancer cell is from a cancer selected from the group consisting of melanoma, lung carcinoma, colorectal adenocarcinoma, lymphoblastic leukemia, lung carcinoma, chronic myelogenous leukemia, promyelocytic leukemia, glioblastoma, breast carcinoma, ovarian carcinoma, prostate carcinoma, head and neck squamous cell carcinoma, and laryngeal squamous carcinoma.

47. The method of claim 44, wherein the cancer cell expresses higher levels of SCC-S2 mRNA than an equivalent non-cancer cell.

48. The method of claim 44, wherein the cancer cell is from a cancer selected from the group consisting of melanoma, lung carcinoma, colorectal adenocarcinoma, lymphoblastic leukemia, lung carcinoma, chronic myelogenous leukemia, promyelocytic leukemia, glioblastoma, breast carcinoma, ovarian carcinoma, prostate carcinoma, head and neck squamous cell carcinoma, and laryngeal squamous carcinoma.

49. The method of claim 47, wherein the cancer cell is from a cancer selected from the group consisting of melanoma, lung carcinoma, colorectal adenocarcinoma, lymphoblastic leukemia, lung carcinoma, chronic myelogenous leukemia, promyelocytic leukemia, glioblastoma, breast carcinoma, ovarian carcinoma, prostate carcinoma, head and neck squamous cell carcinoma, and laryngeal squamous carcinoma.
